(19) United States
(12) Patent Application Publication
(10) Pub. No.: US 2021/0085846 A1
(43) Pub. Date: Mar. 25, 2021

(54) VENTRICLE ASSIST DEVICE

(71) Applicant: Maliel Holding, Oslo (NO)

(72) Inventors: Peter AYRE, Frenchs Forest (AU); John BEGG, Fitzroy Falls (AU)

(21) Appl. No.: 17/109,185

(22) Filed: Dec. 2, 2020

Related U.S. Application Data
(62) Division of application No. 16/318,226, filed on Jan. 16, 2019.

(30) Foreign Application Priority Data
Jun. 1, 2016 (AU) 2016902113
May 8, 2017 (AU) 2017901685

(51) Int. Cl. A61M 1/10; F04D 29/22; F04D 29/24; F04D 29/42; A61M 1/12; F04D 13/02; F04D 29/041

(52) U.S. Cl. CPC A61M 1/1036; F04D 29/2222; F04D 29/242; F04D 29/4273; A61M 1/1013; A61M 1/1031; A61M 2205/8206; F04D 13/027; A61M 1/1012; F04D 29/041; A61M 1/1015; A61M 2205/32; A61M 1/122

(57) ABSTRACT

A ventricle assist device comprising a device body with a housing having an inlet and an outlet. A centrifugal pump is disposed in a portion of the housing. The inlet is adapted to allow a flow of blood into the device body housing and an outlet adapted to allow the flow of blood from the device body housing. The flow of blood from the device body housing is primarily directed into the left ventricle, and the inlet and the outlet are positionable in a ventricle.

### CROSS REFERENCE TO RELATED APPLICATIONS

This application is a divisional of U.S. application Ser. No. 16/318,226, filed Jan. 16, 2019, which is the U.S. national phase of International Application No. PCT/AU2017/050503 filed May 29, 2017 which designated the U.S. and claims priority to AU Patent Application No. 2016902113 filed Jun. 1, 2016 and AU Patent Application No. 2017901685 filed May 8, 2017, the entire contents of each of which are hereby incorporated by reference.

### TECHNICAL FIELD

The present invention relates to a heart assist device which may improve the flow of blood through a user. More particularly, the present disclosure is directed towards a device which may be partially installed in a heart of a user which may assist with pumping blood through the vascular system of the user.

### BACKGROUND

There are a number of devices which assist with improving the blood flow of a patient. These devices can be total artificial heart (TAH) devices, ventricular assist devices (VAD), artificial cardiac pacemakers and cardiopulmonary bypass machines.

AH devices can be used to completely replace a patient heart, however these devices are a last resort as the patient is still waiting to receive a heart transplant.

Artificial cardiac pacemakers are designed to regulate the pulse of a heart and are and generally comprise an array of electrodes attached to the heart in which the electrodes cause the heart muscles to contract at predetermined intervals.

Cardiopulmonary bypass (CBP) machines are used to temporarily assume the function of a heart and lungs during surgery. These devices are not suitable for implantation and are only suitable for use as an extracorporeal medical device.

VADs are designed to assist a failing heart by either partially replacing or fully replacing the function of a portion of the heart, such as a failing ventricle. There are commonly three types either a left ventricle assist device (LVAD), a right ventricle assist device (RVAD) or a combination of right and left ventricle assist (BiVAD). These devices are an electromechanical circulatory device and are often used to keep a patient's quality of life relatively high.

However, these devices have a number of problems which may reduce the potential for a patient's quality of life. As such, the present disclosure may provide for a device which may improve the quality of life of a patient.

Previously, there have been many attempts to create an improved heart assist device. Specifically, many of the previous inventions in this field have focused on providing a left ventricular assist device which is implantable. However, these devices are generally cumbersome or cannot provide for a reliable device to sustain life. Further, these devices generally consider certain types of fluid flow to be a disadvantage.

Most of the devices and systems that have targeted the permanent implant market have focused on developing blood pumps that are suitable for beyond the general average life expectancy of the patient. This leads many implantable Left Ventricular Assist Devices (LVADs) to be too complex for purpose and being extremely expensive to manufacture.

Many of the LVADs used for permanent implantation are manufactured from stainless steel, nitinol, or titanium alloys. All of these exotic metals are relatively expensive to machine or mill and difficult manufacture.

Additionally, there have been many previous inventions that target short term usage (typically less than 6 hours) and are typically not implantable. Also these inventions tend to be only suitable for applications during heart bypass operations or similar emergency situations. A majority of these types of devices are constructed of polymeric materials. A majority of these devices are designed to provide maximum pumping efficiency of the pumping fluid. However, many of these types of devices fail to reduce shearing forces on the pumping fluid. In LVADs, the pumping fluid is typically blood and wherein the LVAD imparts a relatively high shearing force on the blood, the blood tends to clot or haemolyse.

The previous short term devices typically result on patient complications or serious adverse events occurring for usage extending beyond about 8-12 hours. Also many of these short devices rotate at higher relative levels of rotations per minute (RPM) than the longer term devices and this may further exasperate the haemolysis effect.

U.S. Pat. No. 7,862,501—Woodard discloses a pumping system for assisting the circulatory system of a patient, wherein the system includes a rotary flow blood pump by a first cannula connected to a portion of the left side of the heart and a second cannula connected to the aorta; and characterised in that the pumping speed of said pump is adjusted in accordance with measurements from a pressure sensor mounted in or on an inner wall of a portion of the left side of the heart. However, there are a number of issues with this device in relation to the flow of fluid through the device to be delivered to a desired location in the heart. Further, the size of this device is large and cannot be mounted in a heart and there are a number of difficulties even mounting this device in vivo. These devices are also expensive to manufacture.

U.S. Pat. No. 6,609,883—Woodard et al describes a blood pump fabricated mainly from Titanium-6 Aluminum-4 Vanadium (Ti-6Al-4V) coated with amorphous carbon and/or diamond-like coatings. In particular, the pump housing of this blood pump is metallic and includes a magnetic drive motor acting on a hydrodynamic impeller within the pump housing. One of the disadvantages with this invention is that as the pump housing is entirely constructed of metal, electrical eddy currents form between the motor stators and permanent magnets positioned within the impeller. These electrical eddy currents significantly reduce the electrical efficiency of the blood pump and may lead to increased power consumption. Further, this device is of a size too large to be easily implanted and may not achieve a desired blood flow.

Another U.S. Pat. No. 6,158,984—Cao et al describes a modified blood pump in which structural members are inserted within the pump housing between the motor stators and the impeller. These structural members are constructed of a biocompatible, corrosion resistant, electrically non-conductive (insulative) ceramic material. One of the disadvantages with the structural members being comprised of ceramic material is that ceramic material is relatively expensive and difficult to construct. The ceramic material may include a diamond like coating which may be particularly costly to produce and prone to flaking. Further, this device has a number of stagnant blood locations which may adversely impact the viability of this device when used in a patient. Thrombogenesis events may also occur in the implant device near the journal bearing.

Another US Patent Application 20070270633—Cook et al describes a centrifugal blood pump with a hydrodynamically suspended polymeric impeller. This device includes an impeller of a difficult manufacturing shape with dimensional stability issues relating to the tights tolerances of the impeller blades in relation to the housing. Minor dimension changes in use or in moulding of this invention may possibly lead to pump stop or clotting issues. This device may not be able to achieve a desired flow of fluid consistently and suffer pump stop if the impeller deforms.

It has been previous known to this field, that rotary blood pumps may be entirely constructed from polymeric material except for the motor components. However, pumps that are entirely constructed of polymeric materials may lack the desired: wear resistance or strength, fluid impermeability and bio-resistance necessary for this type of application. These types of pumps commonly warp or distort due to fluid absorption limiting their usefulness. This device may additionally, have fluid flow issues which may lead to clotting or coagulation.

Any discussion of the prior art throughout the specification should in no way be considered as an admission that such prior art is widely known or forms part of common general knowledge in the field.

### SUMMARY

### Problems to be Solved

The present disclosure may provide for an improved heart assist device.

The present disclosure may provide for an improved pulsatile heart assist device.

The present disclosure may provide for a device which allows for an effective heart assist device.

The present disclosure may allow for a more effective device for assisting with the flow of blood.

The present disclosure may allow for a superior flow of blood of a user in the ventricle of a heart.

The present disclosure may allow for a flow of blood in a ventricle to be directed to an aorta of the heart via a ventricle.

The present disclosure may allow for a device which may improve the quality of life of a patient.

It may be advantageous provide for a generally low cost or easier to manufacture LVAD wherein the risk of haemolysis or blood clotting is relatively reduced or minimised.

It may be advantageous to provide for a means which may impart a vortical flow to a fluid in vivo.

It may be advantageous to provide for device which allows for a means of transferring between a turbulent flow to a laminar flow.

It may be advantageous to provide for a device which may expel a fluid in a generally irrotational flow.

It is an object of the present invention to overcome or ameliorate at least one of the disadvantages of the prior art, or to provide a useful alternative.

### Means for Solving the Problem

A first aspect of the present disclosure may relate to a ventricle assist device. The device may comprise a device body with a housing, an inlet and an outlet. A centrifugal is pump disposed in a portion of the housing. The inlet adapted to allow a flow of blood into the device body housing and an outlet adapted to allow the flow of blood from the device body housing; and wherein the flow of blood from the device body housing is primarily directed into the left ventricle, and the inlet and the outlet are positionable in a ventricle.

Preferably, an impeller of the drive unit is at least partially positioned in the ventricle. Optionally, the device may cause a pressure differential in the ventricle. Preferably, the pressure differential may be adapted to direct a flow of blood towards the aorta. Preferably, the inlet is disposed relatively perpendicular to the outlet. Optionally, a relative distance between the inlet and the outlet may be at least 10 mm. Preferably, an upper end of the housing is conically tapered to the inlet. Preferably, a battery is disposed in the housing. Preferably, the device can effect a vortical flow adjacent to the inlet. Preferably, the device may be adapted to eject a laminar flow of fluid. Preferably, the housing comprises an impeller housing and a drive unit housing. Preferably, the drive unit housing may be adapted to house a drive unit of the centrifugal pump. Preferably, the impeller housing may comprise an impeller of the centrifugal pump. Preferably, the outlet is directed towards the apex of the ventricle. Preferably, the impeller may comprise a radiopaque marker.

Another aspect of the present disclosure may relate to a ventricle assist device, the device. The device comprising a device body with a housing, an inlet and an outlet and a centrifugal pump disposed in a portion of the housing. The inlet adapted to allow a flow of blood into the device body housing and an outlet adapted to allow the flow of blood from the device body housing, and wherein the flow of blood into the inlet is a vertical flow of blood from the atrium and to be ejected into the descending aorta.

In the context of the present invention, the words “comprise”, “comprising” and the like are to be construed in their inclusive, as opposed to their exclusive, sense, that is in the sense of “including, but not limited to”.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

FIG. 1 illustrates an isometric view of an embodiment of the device of the present disclosure;

FIG. 2 illustrates a perspective view of an embodiment of the device of the present disclosure;

FIG. 3 illustrates an embodiment of the device of the present disclosure with a portion of the impeller housing removed;

FIG. 4 illustrates a perspective view of an embodiment of the device of the present disclosure with a further portion of device housing removed;

FIG. 5 illustrates a perspective view of an embodiment of the impeller of the device of the present disclosure and a base plate;

FIG. 6 illustrates a perspective view of an embodiment of the device of the present disclosure;

FIG. 7 illustrates a perspective view of an embodiment of the impeller housing of the present disclosure;

FIG. 8 illustrates an exploded view of an embodiment of the device of the present disclosure;

FIG. 9 illustrates a cross sectional view of an embodiment of the device of the present disclosure;

FIG. 10 illustrates an embodiment of the device of the present disclosure and a possible blood flow which may be achieved by said device;

FIG. 11 illustrates a further embodiment of the device which is used to bypass a mitral valve such that blood can be directed from the atrium through the device and into the ventricle;

FIG. 12 illustrates yet another embodiment, of the device of the present disclosure;

FIG. 13 illustrates a top perspective of an embodiment of an impeller with a volute shaped based plate; and

FIG. 14 illustrates a top view of a further embodiment of an impeller with a volute shaped based plate.

### DESCRIPTION OF THE INVENTION

Preferred embodiments of the invention will now be described with reference to the accompanying drawings and non-limiting examples.

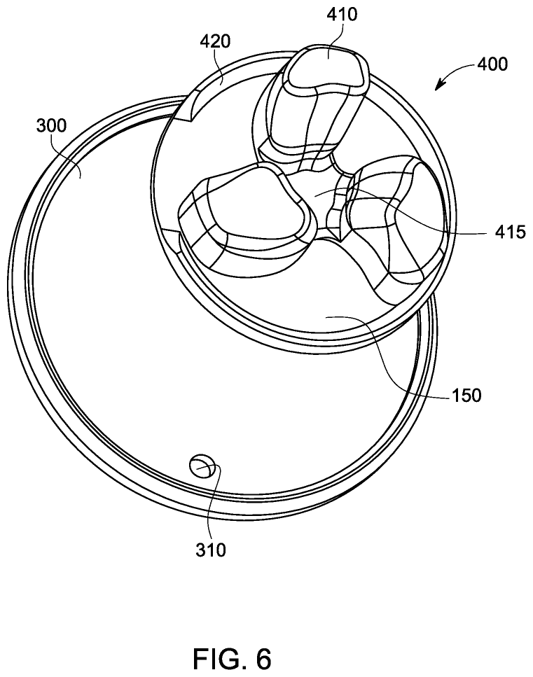

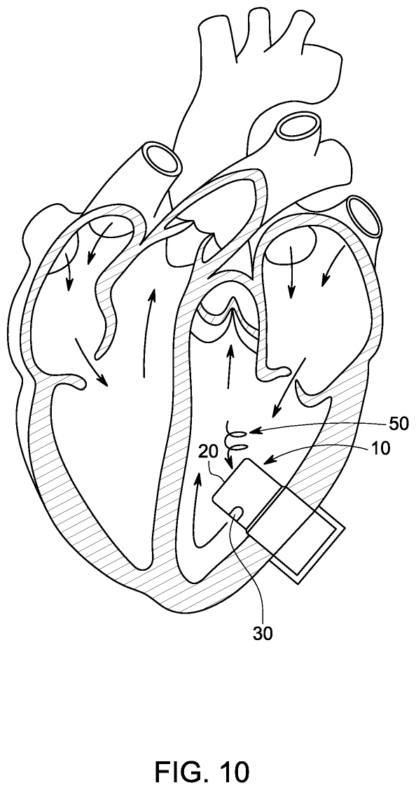

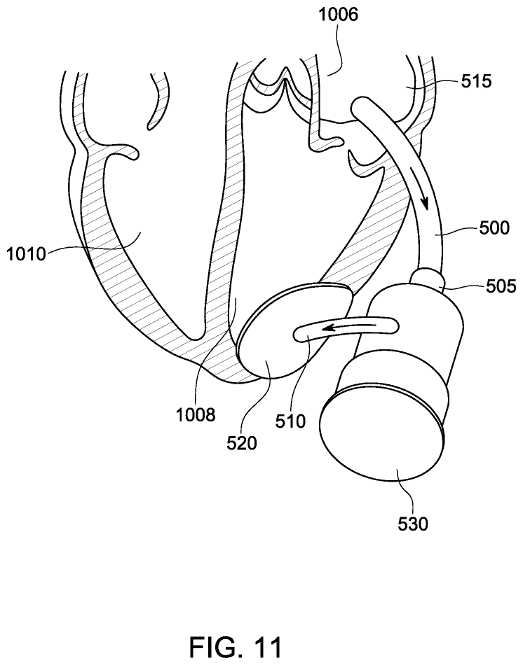

The present disclosure is directed towards a heart assist device, and more particularly a ventricle assist device (VAD). The device 10 is preferably mounted at least partially in a ventricle of a user and at least partially through the myocardium of the user. Preferably, the device 10 is adapted for use in the left ventricle. A power source may be connected to the device 10 to power a hydrodynamic flow means 400 by implanted battery pack or extracorporeal power lead. A power lead (such as the extracorporeal power lead) may connect the drive unit to a battery pack, wherein the power lead extends through aperture 310 (see FIG. 6, for example). The hydrodynamic flow means 400 is preferably a means used to impart a desired flow to a fluid, such as blood, through the device 10, but may also impart a desired motion or flow to a fluid external to the device (see FIGS. 10 and 11). The hydrodynamic flow means 400 may also refer to the shape of the impeller housing 100 as this may also impart a desired fluid flow.

Preferably, the hydrodynamic flow means 400 is an impeller 400. The impeller 400 is manipulable via non-physical means, such as a magnetic levitation means or other magnetic manipulation means. The impeller 400 may comprise encapsulated and sealed passive or permanent magnets. The impeller 400 is preferably housed in the impeller housing 100. The impeller may be in communication with a drive unit 450.

Preferably, the impeller 400 may function as a mixed flow centrifugal pump. The centrifugal pump 400 may be adapted to rotate to cause a desired flow effect on blood in at least a portion of a heart. The desired flow effect may be a vortex or other fluid effect in which blood in a heart, preferably a ventricle of a heart, is drawn into the device impeller housing 100. Commonly, a vortical flow (a vortex flow) is undesirable as this may reduce the efficiency of the device, however the device of the present disclosure may function with a desired efficiency and may be adapted to impart a vortical flow to fluid near to the inlet 20 or the upper portion of the hour glass shape 110 of the impeller housing 100.

The device 10 impeller housing 100 may have a tapered or hourglass type shape at the inlet, and the outlet may be an opening adapted to allow the flow of blood back into the ventricle. The hourglass shape inlet (comprising 110, 20 and 120A) may impart a desired flow to a fluid external to the device 10 which is then drawn into the device 10. The hourglass shape (110, 20 and 120A) may impart a pressure differential between the upper portion of the hourglass 110, through the inlet and into the cavity 120 of the impeller housing 100. The hourglass upper portion 110 may be substantially the same as a lower portion of the hourglass 120A. The inlet maybe bevelled, rounded or shaped to allow for a flow of fluid substantially without impedances. The hour glass shape may be two frustums 110, 120A which meet at inlet 20.

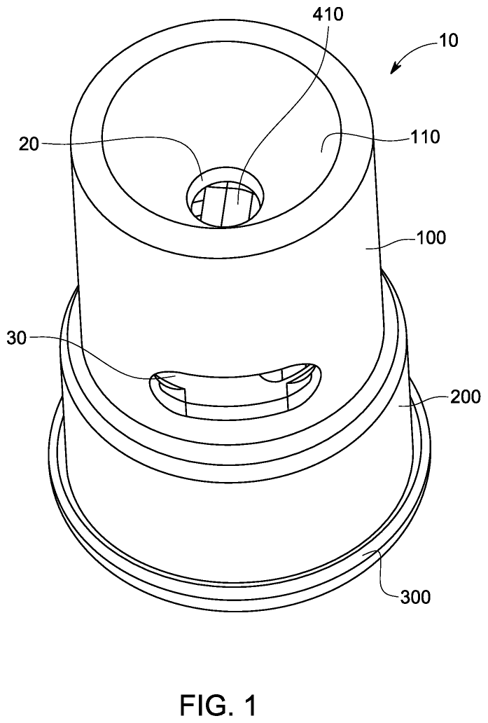

Referring to FIG. 1 there is shown an embodiment of the device 10 with an inlet 20 and an outlet 30. An impeller housing 100 is connected to drive housing 200 in a fluid tight manner. The fluid tight manner may be achieved by an adhesive, a seal, a gasket, welding, ultrasonic welding or any other sealing method.

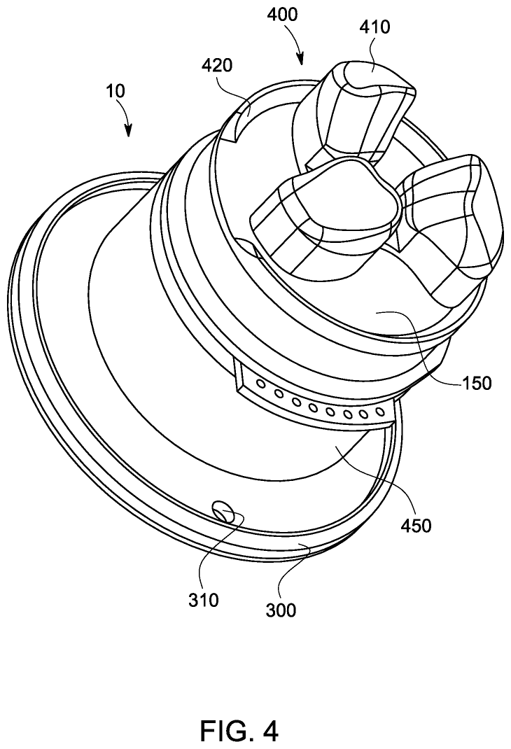

Referring to FIGS. 4, 8 and 9, the drive unit 450 is housed in the drive housing 200 and drives the hydrodynamic flow means 400. The hydrodynamic flow means 400 is illustrated as three blade impeller 400. The blades 410 of the impeller 400 are adapted to cause a suction of blood, in use, such that blood is drawn through inlet 20 and into the cavity 120 of the impeller housing 100. The centrifugal nature of the impeller 400 may then force blood out the outlet 30 of the device 10. The impeller housing 100 is preferably shaped such that a desired fluid flow is imparted to blood. For example, a vortex blood flow may be advantageous, a suction flow or any other predetermined flow.

Each of the blades 410 is preferably formed with a similar shape to allow for a desired flow of fluid through the device 10. However, while it is preferred that the impeller blades 410 are of substantially the same shape, at least one of the impeller blades 410 may be formed with a different shape or a protrusion to impart a desired flow of fluid through the device. For example, if a first impeller blade 410 is of a shape which is larger than that of the other two blades 410, the first impeller blade 410 will come into contact with fluid passing through the device before the other two blades 410, which may assist with forming a desired flow of fluid. If a shape of the blade 410 is changed, the remaining blades 410 may have a weight or other mass applied such that the impeller 400, in use, is balanced. Further, the shape and/or rotation of the impeller blades 410 are such that shear stress on the blood cells is reduced to prevent damage to blood. The outlet 30 straightens the flow so that blood exiting the impeller housing 100 enters the ventricle and is directed towards the apex of the ventricle and subsequently to the aorta (See FIG. 10). In the case of an anastomosis device 10 (a device which is external to the heart as seen in FIG. 11), the blood flow from the device 10 is directed either towards the apex or the aorta.

The blood is preferably drawn into the device 10 axially through the inlet 20, with the blood preferably moving in a vortical manner or with a rotational flow. The vortical manner or a rotational flow preferably flows around an axis defined by the axial direction of the device 10. Permanent magnets are enclosed in the center of the impeller 400 or impeller blades 410 and two motor windings are located in the casing on each side of the rotor. This configuration preferably comprises a DC brushless motor; this is a simple and durable motor which minimises the number of mechanical parts. The power cable is connected directly to the DC brushless motor controller to change motor speed.

The impeller 400 preferably allows for a reduction in a haemolysis rate by decreasing the time of exposure of the blood to friction forces and by reducing the intensity of these forces.

The impeller 400 forms a portion of a mixed-flow centrifugal pump which may be magnetically driven to rotate. Preferably, the rotation of the impeller is preferably clockwise (when viewed from the top of the inlet 20). The device 10 includes a housing, impeller housing 100, drive housing 200 and drive housing base plate 300. The impeller 400 and the drive unit 450 may be collectively referred to as a “pump” or “blood pump”. Each of the housings 100, 200 having an upper and lower portion and an internal cavity for housing components of the pump, such as the impeller 400 and the drive unit 450. Preferably, a plurality of impeller blades 410 are housing in the cavity of the impeller housing 100, in which the impeller blades 410 are adapted to rotate within the impeller housing cavity 120. While there is illustrated an embodiment in the Figures comprising a plurality of impeller blades 410, the impeller may optionally comprise a single impeller blade 410 (not shown). If a single impeller blade 410 is used, the base plate 150 may be weighted to allow for a desired balance of the impeller 400. When the impeller 400 rotates it imparts a centrifugal force on the blood which occupies the cavity when in use. The centrifugal forces are preferably of such a nature that they minimise the shear forces acting on the blood cells such that the blades do not cause undue damage.

The rotation of the impeller preferably effects a cyclonic flow or vortical flow of the blood near to the inlet of the device 10. Preferably, only the blood at the location axially the inlet is imparted with a cyclonic or vortical flow (see FIG. 10). While FIG. 10 illustrates an anti-clockwise flow, the device 10 may be adapted to allow for a clockwise flow of blood into the device. Effecting a vortical flow provides for a suction of blood into the device via the inlet 20 while also being able to expel blood from the outlet 30 of the impeller housing 100. A base plate 150 is provided in which the impeller 400 can be mounted. Preferably there is a gap between the base plate 150 and the impeller 400 such that frictional forces are reduced, thereby improving the efficiency of the device 10.

In use, as the impeller rotates, blood is forced or pushed in a radial direction away from the centre of the impeller housing 100 towards an outer wall of the impeller housing 100. As the blood rotates, it eventually exits the impeller housing 100 through the outlet 30. Thereby the rotation of the impeller 400 effects flow of the blood from the inlet 20 the outlet 30. The ejection of the blood from the device 10 is preferably directed towards the apex of the ventricle. Centrifugal blood pumps of this configuration may generally reduce shearing forces on blood.

The size of the device 10 is such that at least a portion of the device 10 is implantable into a ventricle of the heart. The device 10 may be adapted to be used with the right or the left ventricle. More preferably, the device 10 is adapted for use with the left ventricle of the heart. This small sizing may produce optimal pumping conditions wherein the impeller RPM is not too high to cause significant levels of blood damage. For example, the frequency of the impeller may be adapted to be in the range of 1,000 RPM to 10,000 RPM, but more preferably the RPM of the impeller 400 is 1,500 RPM to 5,000 RPM. Due to the imparted flow of the blood into the inlet 20 or impeller housing 100 and/or the shape of the inlet 20, the device may reduce the risk of haemolysis or coagulation of the blood in the device 10. Further, the ejection of blood to the apex of the ventricle may assist with a reduction of stagnant blood or the “dwell time” of blood in the heart.

At the perimeter of the base plate 150 a projection 420 is formed which may assist with mating the impeller housing 100 to the base plate 150. The projection 420 may provide for a seal to be formed between the impeller housing 100 and the base plate 150 to prevent fluid, such as blood, passing between the impeller housing 100 and the projection 420. The projection 420 may be angled or formed to act as a volute to impart a desired flow of fluid within the impeller housing 100. Optionally, the impeller housing 100, can be formed to act as a volute is desired. In a further embodiment, the projection 420 is uniform near to or at the periphery of the cavity such that the cavity is devoid of a volute.

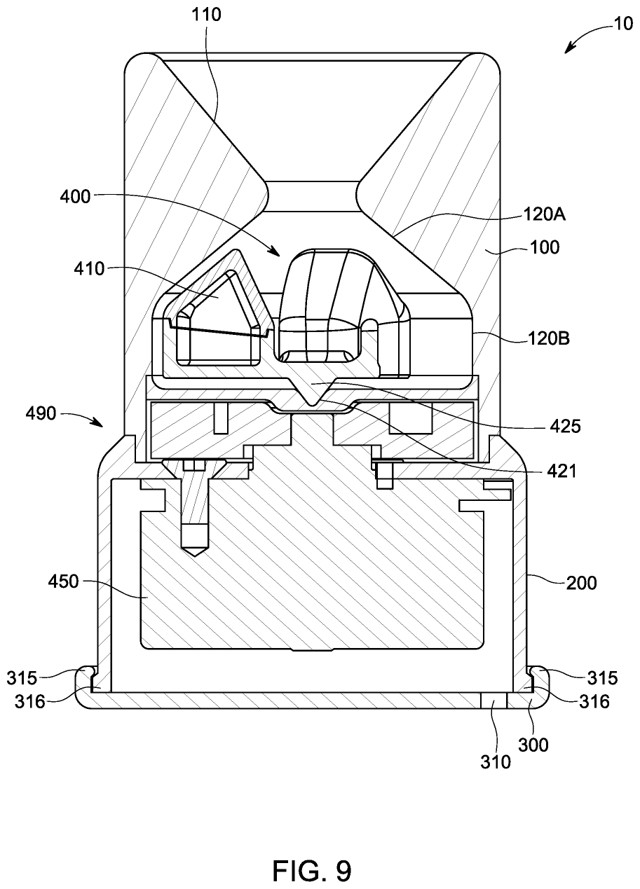

The impeller is preferably positioned near to the middle of the base plate towards the centre of the cavity 120 when the impeller housing 100. The impeller blades 410 may be hollowed out to receive a magnet or an element which is influenced by a magnetic field such that a magnetic field means, (e.g. a magnet) in the drive housing 200 can impart a rotation or movement to the impeller 400. A column may be provided in which the impeller 400 may be adapted to spin about the axis of the column. Preferably the impeller 400 includes a recess (not shown) adapted to receive the central column and/or the base plate comprises a base recess 421 to receive the column. Preferably, the central axis of the column acts as a pivot bearing 425 which in turn engages with the recess of the impeller 400 and/or the recess of the base plate 150. Referring to FIG. 9, there is illustrated a pivot bearing 425 which is adapted to engage with a recess 421 of the base plate 150, similar to that of a spindle. Optionally, the column pivot bearing 425 may be replaced with a ball bearing or other predetermined bearing which allows for the rotation of the impeller 400 relative to that of the base plate. Optionally, the bearing for the impeller 400 may be a thrust bearing or a journal bearing (not shown).

When in use, the impeller 400 rotates about the central column. The pivot bearing 425 is mounted in the middle or centre of the uppermost point of the central column and is preferably constructed of a low wear resilient and biocompatible material such as titanium alloy, stainless steel or ceramic. Preferably, the pivot bearing is in the form of a single ball bearing (not shown), the cost of manufacture of this component is relatively low cost to manufacture compared with other bearing assemblies. This may allow for a lower cost VAD to be manufactured. The act of rotation of the impeller 400 imparts a stabilisation force on the impeller 400, wherein the impeller 400 experiences forces at approximately 90 (ninety) degrees to the axis of rotation. The stabilisation force is relatively constant around the outer circumference of the impeller 400 which provides for a desired flow effect to be generated within the impeller housing 100 and proximal the inlet 20 of the device 10.

Preferably, the impeller 400 is constructed from polymeric materials that are biocompatible and resist to fluid ingress. Constructions materials may include PEEK, polycarbonate (PC) or polyurethane (PU). However, metal alloys such as stainless steel or titanium alloy may optionally be used. Preferably, the impeller may include magnets mounted or positioned within the blades 410.

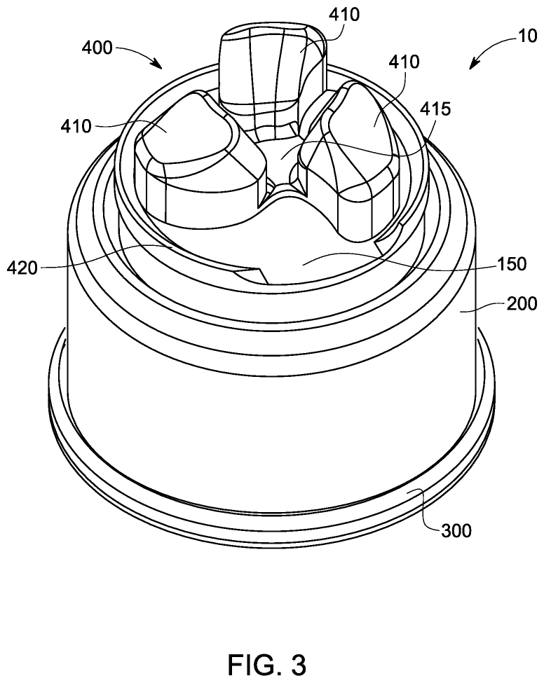

Preferably, the impeller 400 as depicted in FIGS. 3 to 6 includes three impeller blades 410 extending radially from a central connection point (see FIGS. 3 and 6, for example). Preferably, the underside central connection point is formed with a recess for a bearing, that is if a recess if required in the impeller 400. The blades 410 each respectively have a general triangular profile 410A or wedge shape profile when viewed from the top view. The number of blades 410 may be varied so long as the impeller 400 remains balanced when in use or rotation.

Each blade 410 is preferably connected to its neighbouring respective blade by an arm or bridge 415. Preferably, the impeller 400 when viewed from the top view in FIG. 3 has an overall triangular appearance when three blades 410 are utilised in the design. However, other variations may comprise four blades 410 forming square shaped impeller 400 or five blades 410 forming a pentagon shaped impeller 400, or any other predetermined number of impeller blades 410. Three blades 410 are preferred in the preferred embodiment as reduces the amount of machining required for production of the blades 410 and also reduced the amount of material required to manufacture the impeller 400. In addition, due to the size of the device 10 being relatively smaller than most known VAD devices having three impeller blades 410 are advantageous. Having fewer blades 410 also may reduce the shear stress imparted to fluid and reduce impact damage to fluid flowing the in the impeller housing 100, thereby reducing blood clotting or haemolysis.

The impeller 400 is preferably driven to rotate by the interaction and cooperation of sets of magnets. Preferably, a drive unit 450 which is housed in drive housing 200 includes an electrically actuated motor (which may preferably be a DC brushless motor) mechanically connected to a pivot member 460. The pivot member 460 may be integrally formed with the shaft of the motor within the drive unit 450. The pivot member 460 may include a first set of permanent magnets mounted, positioned or integrally formed with the outer surface of the pivot member 460. Wherein the motor is actuated, the shaft and elongated pivot member 460 may also be rotated in the desired direction. The motor preferably imparts a movement of the impeller in the direction of a concave surface of the impeller blades 410. The pivot member 460 may be housed in the drive housing 200.

A second set of permanent magnets are to be mounted, positioned or integrally formed with the blades 410 in the impeller 400. The impeller blades 410 may be formed with two portions, an upper portion 410A and a lower portion 410B, in which at least one of the portions comprised a receptacle in which a magnet, a material which is influenced by a magnetic field or weight may be disposed. The upper and lower portions 410A and 410B may be welded, adhered, fixed or sealed such that fluid is prevented from entering the receptacle. The second set of permanent magnets is adapted to magnetically engage with the respective magnets forming the first set. When the first set of magnets are rotated by the motor, the second set of magnets will transfer torsional force to the blades 410 and rotate the impeller 400. It will be appreciated one of the first set and the second set of magnets may optionally instead be a material which is influenced by a magnetic field such that both the first and second sets of magnets are not magnets.

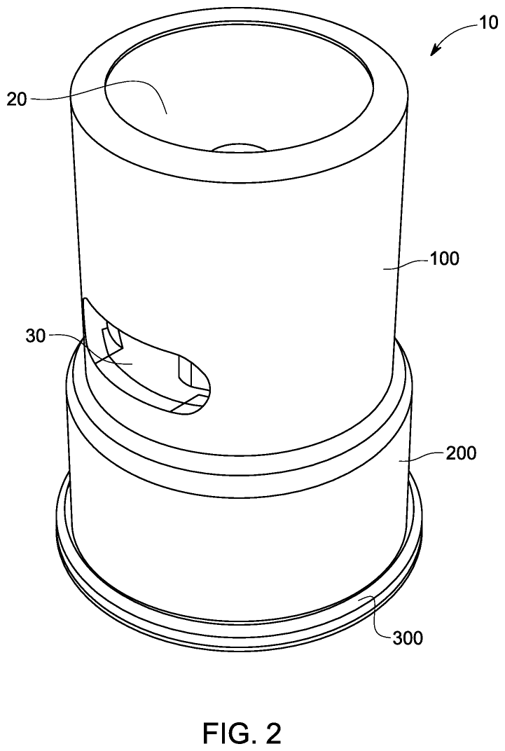

Further, the attractive forces between the first and second sets of magnets are adapted to apply a subtle downward pressure (relative to the side view shown in FIG. 2) on the impeller 400 to ensure that the impeller 400 does not lift off from the pivot bearing. This may form a limited magnetic restraint in the movement of the impeller 400 in the vertical axis away from the base plate. Thereby, the impeller 400 is preferably suspended in the cavity 120 by a combination of at least two of; a magnetic force, a pivot bearing applying a physical force upwardly and a centrifugal force of the blades 410, when in use.

Preferably, the impeller housing 100 and the impeller 400 are constructed of polymeric materials except for the drive unit 450 and the sets of permanent magnets. Preferably, the permanent magnets are constructed of rare earth magnets and these magnets may be coated and encapsulated with an impermeable substance to prevent fluid ingress or corrosion of the magnets. The coating and/or encapsulation is preferably formed from a biocompatible material such that fluid is allowed to enter the drive housing 200, the components inside said drive housing 200 do not adversely impact the user of the device 10. Alternately, polymer or plastic magnets may be used as permanent magnets. These polymer magnets are non-metallic magnet and resistant to corrosion and made from organic polymer. An example of a suitable organic polymer may be PANiCNQ which is a combination of emeraldine-based polyaniline (PNAi) and tetracyanoquinodimethane (TCNQ). These polymeric materials may be influenced by magnetic fields.

Preferably, the drive housing 200 preferably includes mating means portion tongue 316 extending from the lower portion of the impeller housing 100 in a radial direction away from the centre of the drive housing 200. The drive housing 200 is adapted to mate or be secured with a drive unit housing base 300. The drive housing 200 preferably comprises a motor and components thereof. Preferably, a further mating means portion, flange or lip 315 is adapted to engage the lower portion of the drive housing 200 at the tongue 316. As shown in FIG. 9, the flange has engaged the lower most outer surface of the drive unit 200 and acts to seal the drive unit 450 in the drive housing 200. Preferably, another mating means may be provided between the impeller housing 100 lower end and drive unit housing 200 upper end. The mating means 490 and/or mating means portions 315, 316 may be resilient and flexible and able to be engaged or disengaged with appropriate hand pressure. At least one of the mating means 490 mating means portions 315, 316 may extend around the full circumference of the impeller housing 100 or the drive housing, respectively. Alternatively, the mating means 490 and/or the mating means portions 315, 316 may be secured just on opposite sides of the drive housing 200.

Preferably, the upper portion of the impeller housing 100 may form a sloping bezel 120A and the lower portion of the cavity 120B is cylindrical or conforms to a shape similar to the outer silhouette of the impeller 400, wherein the upper portion of the cavity 120A is generally conical shaped leading to inlet 20. The impeller 400 may generally include the same or similar conical shape or profile on its upper surface 410A.

Preferably, a pressure sensor (not shown) may be mounted or positioned on the inner wall of the inlet 20. When in use, blood flows from the inlet into the impeller housing 100 past the pressure sensor and an electrical signal is generated by the sensor which may be feedback to a controller which regulates the speed and action of the pump. Additionally, information from the pressure sensor may logged and/or recorded by a controller and supplied to a clinician or physician as the necessary review times.

Referring to FIG. 1, there is illustrated an impeller housing 100 attached and secured to the drive housing 200. The impeller housing 100 having a conical portion 110 which is relatively above the inlet 20.

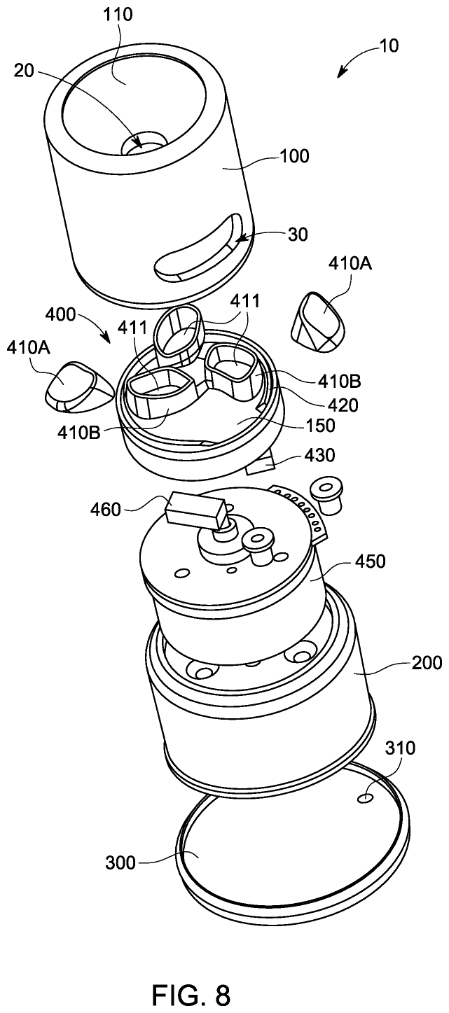

Preferably, the drive unit 450 is electrically attached to a controller by a set of wires (not shown) adapted to commutation control to the motor of the drive unit 450. The wires may be connected to the drive unit 450 via an aperture 310 in the drive unit base 300 (see FIGS. 6 and 8). The drive unit housing base 300 may be connected to the drive housing 200 to house the drive unit 450 therein. The drive unit housing base 300 and the drive housing 200 preferably provides a fluid tight seal for the drive unit 450. Preferably, the controller includes a quick release lever and a socket which cooperates to engage and secure a battery. The power source, such as a battery, used to power the device 10 may be secured to the controller by a releasable locking mechanism such that it can be removed. The power source may comprise Li Ion or NiMH materials.

Preferably, the controller may control the speed of the blood pump by controlling the commutation speed of the motor, and hence the speed of the impeller 400. The speed may be automatically adjusted to suit the needs of the patient, and more preferably the speed is pulsatile such that the impeller speed is modified based on the natural pulse of the heart. The controller may also regulate the pump speed in a pulsatile manner. Alternately, the pump speed may set by a physician and regulated at a suitable level based on feedback from a pressure sensor in the blood pump.

The described system may be partially or fully implantable depending on the circumstances and needs of the patient. The system may also be used to assist the right or left sides of heart. Wherein the system is attached to the right side, the pumping speed is generally lower than that of left side application.

The blades 410 include an upper region 410A and lower region 410B as determined by the top and bottom of the blood pump in which the impeller 400 is mounted or positioned within. The lower region 410B extends generally upwardly in a vertical direction and at about half of the height of the overall blade 410 height, the upper region begins 410A. The upper region is preferably deflected from the vertical axis by an angle of deflection between 1 to 90 degrees (one to ninety degrees). More preferably, the angle of deflection is between 10 to 45 degrees (ten to forty-five degrees). Preferably, the angle of deflection is in a direction opposed to the rotation direction of the impeller 400.

Each of the blades 410 is preferably arcuate or curved when in viewed from a top or bottom view and includes a set of permanent magnets. Preferably, the impeller 400 includes a valley or recess 415 located or positioned between the blades 410 and the hub 415. Preferably, the recess is centred above the hub 415 so as to reduce the risk of haemolysis from slow or stagnant blood flow in the centre of the blood pump.

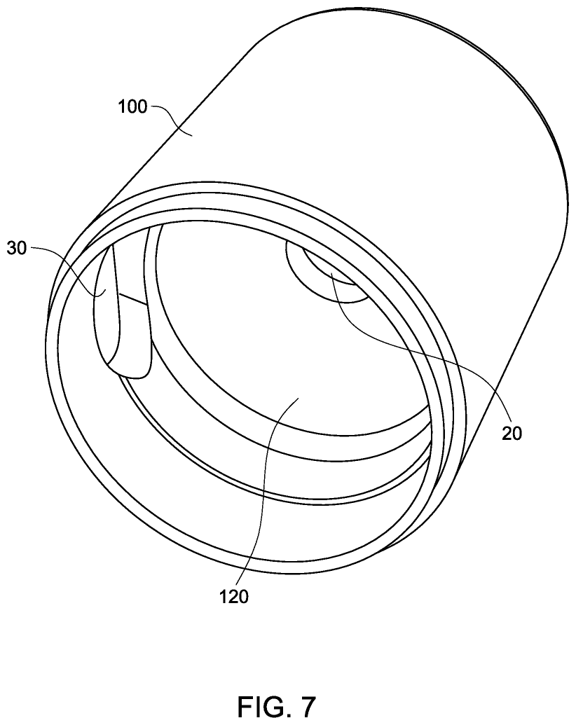

The outer edges of the blades 410 are adapted to conform to the shape of the inner wall or surface of the impeller housing 100, as seen in FIG. 7.

FIG. 9, depicts a pivot bearing recess 421 mounted or positioned in the base plate below the impeller 400, which is adapted to receive a pivot bearing 425 extending from the middle of the impeller 400. The pivot bearing 425 arrangement may include a ceramic or wear resistant pivot bearing to be mounted to allow for the relatively free rotation of the impeller 400 within the housing.

Blood viscosity sensors may also be included within the design and mounted or positioned in the inlet 20 or impeller housing 100 of the pump. Please note that these sensors may be integrally moulded into the polymeric impeller housing 100.

The impeller 400 is generally conical shape wherein the blades 410 extend radially from the hub 415. In yet another embodiment, the controller and battery may be been replaced with a controller bag (not shown). The controller bag preferably includes a controller with an internal rechargeable battery and an external rechargeable battery. The bag is adapted to be portable and carried by the patient holding the strap.

The controller is adapted to communicate with the external PC or hospital monitor. The electrical communication may be achieved by use of Bluetooth™ or Wifi™ interfaces between the hospital monitor and the controller.

Optionally, the controller includes a small internal rechargeable battery which is preferably encapsulated within the same housing as the controller. The controller preferably is connected to the pump and sensors by way a percutaneous lead. The percutaneous lead includes the wiring the power the pump and electrical connections to the sensors within the blood pump.

The controller may also be selectively connected to larger external rechargeable battery. The controller may be adapted to allow for switches between the batteries and to maintain constant power to the pump and sensors.

The power supply may be preferably a mains or AC power supply wherein the power supply provides electricity to the controller and the controller redistributes the current to charge the batteries, when the power supply is connected.

Preferably, the controller may be connected by wire or wireless communication connection to a personal computer (such as a laptop, mobile phone, cell phone, smart phone, notebook, tablet or the like) or hospital monitor. The hospital monitor may be able to download results from the sensors stored within the controller or logged data relating to pump function and speed.

The hospital monitor may be able to backup data from the controller and also display the data in graphical format which is easier for a clinician or doctor to evaluate.

Additionally, the controller may wirelessly interface with other mobile electronic devices such as smart phone or tablet personal computers.

The controller may be adapted to output data to be displayed on a screen or monitor, wherein the screen depicts to the patient, clinician, nurse or doctor basic operating details relating the pump in real time. The displayed data may include graphics depicting various statistics such as battery charge, pump flow, pump pressure, pump output, and wireless connection detection lights.

The device 10 may comprise an internal memory to capture and record a log of pumping, irregularities, sensor electrical stimulations detected, or any other data set detected or monitored by the device 10.

The drive housing 200 is preferably connected to a base plate 300. The base plate 300 further preferably has an aperture 310 or other means to allow cables or other control objects to feed into the device 10. Preferably, the device 10 is powered external to the patient such that power source can be replaced if necessary.

Preferably the housing 100 comprises at least one tapered surface 110 directed towards the inlet 20 and a similar tapered surface may also be seen in the cavity 120, shown as portion 120A (see FIGS. 7 and 9). The tapered surface may impart a desired blood flow or direct blood to a desired direction.

FIG. 2 shows an embodiment of the device 10 similar to the device shown in FIG. 1. FIG. 3 depicts an embodiment of the device in which the housing 100 has been removed and the impeller 400 is shown mounted on the impeller base. The impeller base preferably comprises a tapered wall which may improve the flow of blood when in the cavity 120 of the device 10.

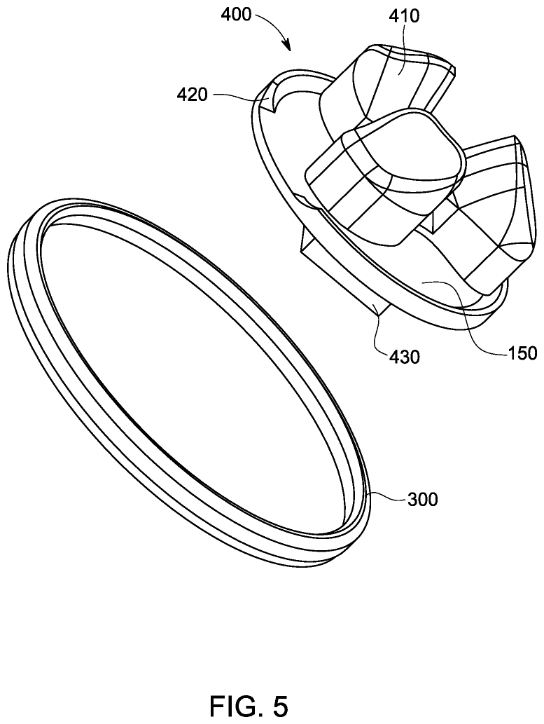

FIG. 4 depicts an embodiment of the device in which the drive housing 200 has been removed and the drive 450 is shown. The drive 450 is preferably adapted to effect movement of the impeller 400. Turning to FIG. 5, there is shown magnets 430 which assist to effect movement of the impeller 400. Effecting movement of the impeller may cause a pressure differential in the ventricle to occur, which may cause an improvement of blood flow relative to the patient's native blood flow in said ventricle.

FIG. 6 illustrates an embodiment of a perspective view of the impeller 400 above the base plate 300. The base plate comprises aperture 310 adapted to allow an element to pass therethrough.

FIG. 7 illustrates an embodiment of the housing 100 with a view of the cavity 120 in which the impeller 400 can be positioned. Preferably, the relatively lower portion of the outlet is substantially planer or flush with the base impeller base, such that there is a reduced potential for coagulation to occur.

FIG. 8 shows an exploded view of an embodiment of the device 10. Preferably, the housing 100 when in use is substantially positioned in the ventricle of the patient, and the drive housing 200 is substantially positioned in the myocardium of the patient.

FIG. 9 is a sectional view of an embodiment of the device in which the housing 100 can be seen with a substantially internal hour-glass shape which may improve the flow of blood through the device. The outlet 30 is not seen in this embodiment. Preferably, the blades of the impeller 400 substantially conform to the shape of the cavity 120.

Referring to FIG. 11, there is illustrated an inflow cannula 500 connected to the left atrium 1006 which leads to the device 10, and then an outflow cannula 510 extends from the device 10 to the left ventricle of the heart. This allows for allows for blood to be pumped from a cored aperture in the left atrium to the left ventricle via a cored aperture in the ventricle, such that as the left ventricle 1008 contracts the blood is urged into the aorta of the heart. The device 10 may also be adapted to function upon insertion into the right ventricle 1010. The outlet 20 of the impeller housing 100 is connected to an outflow cannula 510 which is secured and in fluid communication with the ascending aorta. The cored aperture in the atrium is preferably covered by a pad 515 with is adapted to allow for a cannula to be in in fluid communication with the atrium through pad 515. The pad 515 may be fixed to the outside wall of the heart at the location of the atrium 1006 cored aperture such that the cannula 500 can be attached securely to the heart.

Similarly, a further pad 520 may be disposed on the ventricle to allow covering of the ventricle cored aperture, while still allowing for fluid communication between the ventricle and the cannula 210. Preferably, the respective pad 515, 520 the respective cannula 500, 510 attached thereto have a fluid tight seal such that blood does escape the heart into the body.

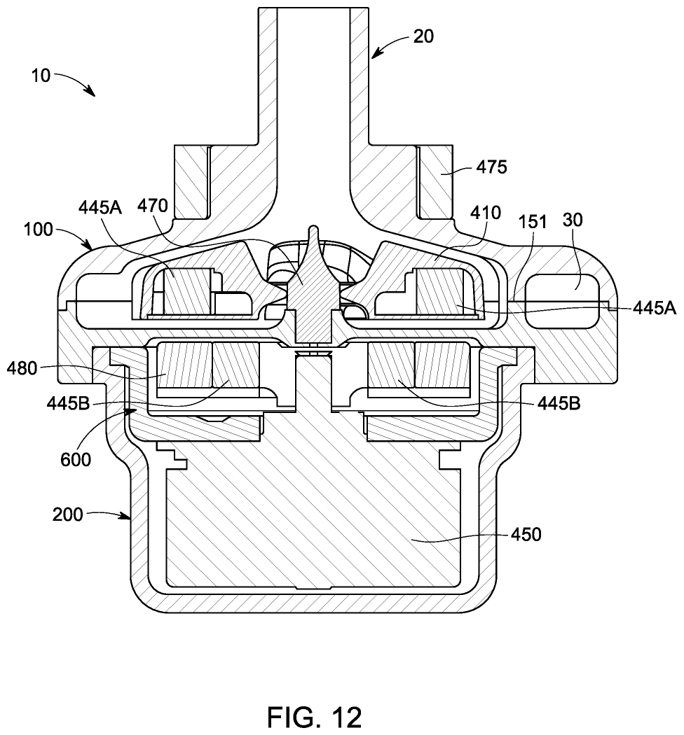

Referring now to FIG. 12, there is illustrated sectional view of a further embodiment of the device of the present disclosure. The device 10 comprises an inlet 20 which is cylindrical or generally tubular which allows fluid to flow into the impeller housing 100. The impeller 400 preferably comprises four blades 410, with each impeller fitted with a magnet 445. The magnets of the impeller 445A may be influenced by at least one of the magnets 445B of the rotor 480 in the rotor housing 600. The rotor housing 600 may be disposed between the drive unit housing 200 and the impeller housing 100. At least a portion of the drive unit 450 may extend into the rotor housing 600 such that movement to the rotor can be effected. The rotor 480 preferably comprises a plurality of magnets 445B which are used to impart a motion to the impeller 400 when the rotor 480 moves or poles of the magnets 445B are altered. Movement of the rotor 480 may be dependent on the drive unit imparting a rotation to the rotor 480 to then impart movement to the impeller. The position of the magnets 445B of the rotor 480 relative to the position of the magnets 445A of the impeller 400 may be offset relatively to each other, as seen in FIG. 12. The polarity of the magnets 445 may be set to oppose in the radial direction or in the tangential direction such that movement may be imparted to the impeller 400 when the drive unit 450 is activated.

The receptacle of an impeller blade 410 may be larger than required to accommodate a magnet 445A. It is preferred to have a receptacle which will snugly, or tightly fit a magnet therein, such that the atmosphere within volume of the receptacle is consumed or substantially filled by the magnet 445A. Reducing the atmosphere or air in the impeller blade may assist with reducing the potential for damage to the magnet, such as corrosion, and reduce the potential for the impeller blade breaking and releasing oxygen into the heart. However, the impeller blade 410 may have additional volume within the receptacle such that the weight of the blades can be controlled to allow for a desired rotational effect in use, which may also save power during use.

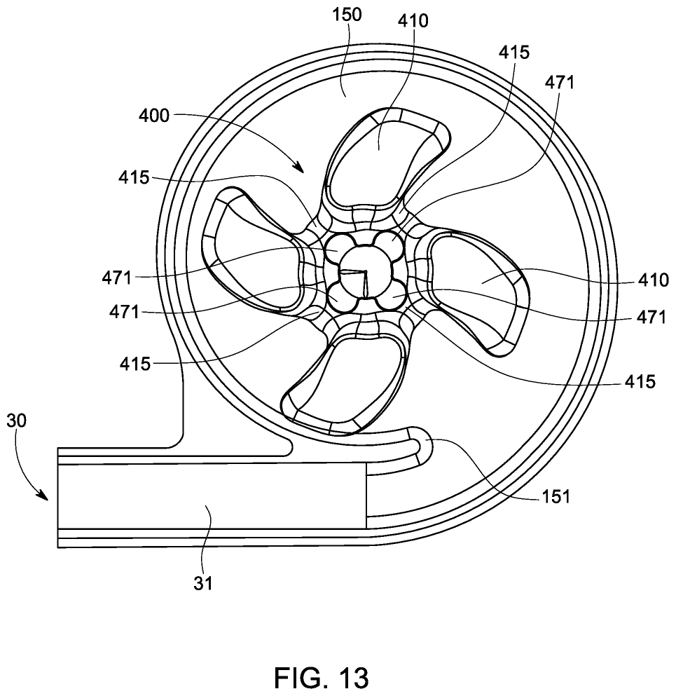

The spindle 470 of the impeller 400 may have a generally bell curved profile shape which allows for fluid entering the impeller 400 cavity to be directed towards the blades 410 of the impeller 400. The spindle 470 and the impeller blades 410 may be separate components such that a first impeller blade 410 is connected, preferably by a bridge 415, to at least one adjacent impeller blade 410 with at least one impeller blade forming an abutting relationship with the spindle 470. The spindle 470 may be adapted to be fixed in position, while the impeller blades 410 rotate about the spindle 470. A plurality of apertures 471 may be defined by the spindle and the bridge 415 portions of the impeller blades 410 as shown in FIG. 13.

Optionally a further magnetic ring 475 or eddy ring 475 is provided such that the magnets 445A of the impeller are positioned between the magnetic ring 475 and the rotor magnets 445B. The ring 475 be used to stabilise the impeller 400 when in use and thereby improving the efficiency of the rotation of the impeller 400.

Referring to FIG. 13 there is illustrated an example of a four blade 410 impeller 400 mounted on a base plate 150 with a spindle in the centre. The spindle 470 in the illustrated embodiment is not connected to the impeller blades 410. The base plate 150 has a shape which, in combination with the impeller housing 100, forms a volute.

A vane or cutwater 151 is provided in the impeller housing 100 and is preferably is connected to the base plate 150. The cut water is preferably a means to allow for diversion of flow preferably towards the outlet 30. At least one of the outlet 30 and the tubular portion 31 may have a flow straightener or vane to influence flow the flow of fluid from the device 10. The flow straighteners may further be disposed at the outlet across the outlet aperture to impart a desired flow to fluid. The outlet 30 may comprise a tubular portion 31 which may urge flow to be a relatively more linear flow or laminar flow when being ejected or leaving the device 10. The cutwater 151 may be rounded or pointed such that a desired flow effect is imparted to the fluid. Rounding the cutwater 151 may allow for a reduction in damage to blood flow in the device during use. The tubular portion may be of any cross-sectional shape, such as a square, a squirkle, a circle, an ovoid, a triangle, a Reuleaux triangle, or any other desired shape. Optionally, the cross section of the tubular portion 31 adjacent the cutwater 151 and the tubular portion 31 near to the outlet 30 comprise different cross sections. For example, the portion of the tubular portion 31 near to the cutwater 151 is preferably generally square or squirkle in cross-section and the tubular portion 31 near to the outlet is preferably circular in cross-section. The cross-section of the tubular portion 31 near to the outlet 30 may be relatively smaller than the cross-section al area near to the cutwater 151, such that the velocity of the fluid leaving the outlet 30 is relatively higher than the fluid velocity relatively near to the cutwater 151.

It will be appreciated that the transition of cross-sections, if there are differing cross-sections, may occur at any location of the tubular portion 31. The transition may be immediate or have a transition region (not shown) which provides for a smoother transition of flow between the cross-sections. The cross-section may also be tapered to increase or decrease flow speeds from the outlet 30. Any number of cross-sectional segments of differing size and/or shape may be used for the tubular portion 31 to impart a desired flow to fluid.

In yet another embodiment, the device may be adapted to allow for pumping and/or flowing of blood from the left atrium to the descending aorta. In this way blood can avoid being injected into the ventricle if the ventricle cannot effectively assist with pumping of blood to the aorta. Pads, similar to that as seen in FIG. 11, may be provided to connect the device 10 to the left atrium and/or to the descending aorta.

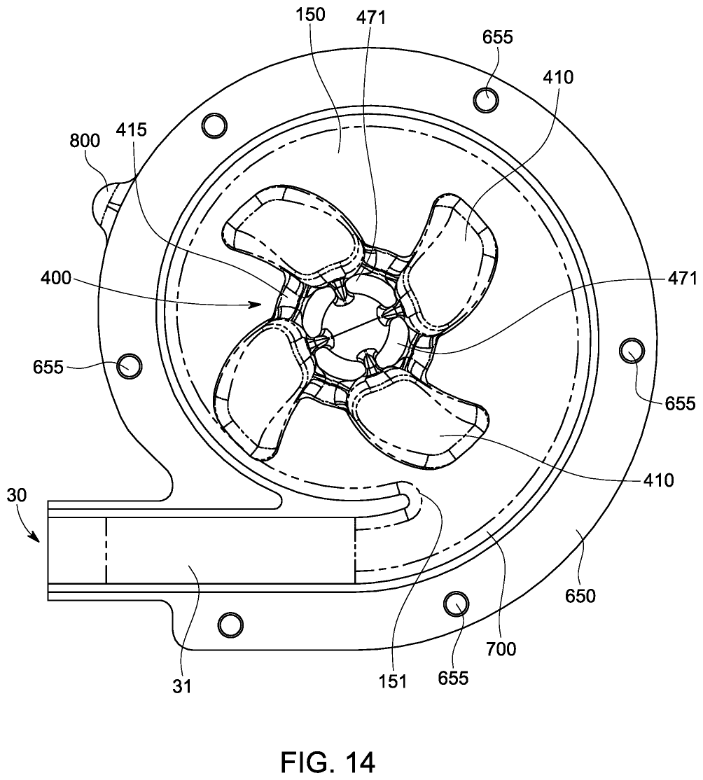

Turning to FIG. 14, there is illustrated another embodiment of the impeller 400 and a volute 700. The rim of the volute 700 may be curved or have a shape which will generally reduce turbulent flow within the impeller housing 100. A periphery 650 drive housing 200 may be relatively larger than the cross section of the impeller housing 100. The periphery 650 drive housing 200 as shown may also have at least one securing means locations 655 to provide a location for the impeller housing 100 to be secured to. The impeller preferably also comprises at least one, but preferably a plurality of securing means locations (not shown), to mate with the periphery 650 drive housing 200. Optionally, a gasket or seal may be provided between the periphery 650 of the drive housing 200 and the impeller housing 100 such that fluid is restricted from accessing sealed areas of the device 10.

The impeller housing 100 and the drive housing 200 may optionally be welded to one another. Welding may be achieved by ultrasonic welding, induction heating or any conventional attaching, welding or fusing processes. This may provide for mating between the housings 100, 200 such that the housings form a unitary structure after welding. However, it will be appreciated that the securing means locations may have simple fasteners mounted therein, such as a nut and bolt, a screw or any other mechanical securing means, or combination thereof.

As illustrated in the embodiment of FIG. 14, a base plate 150 is provided with a volute 700 at or near to the periphery of said base plate 150. The volute 700 may be disposed at only a portion of the periphery of the base plate 150. The volute 700 may improve the flow of fluids or provide a desired flow of fluids within the impeller housing 100.

The impeller 400 in this embodiment comprises a modified spindle 470 in which may provide for improved flow of fluid when fluid enters into the impeller housing. The apertures 471 of the impeller 400 are relatively larger than the embodiment of FIG. 13, which may improve the flow of fluid and minimise stagnation of blood and/or minimise coagulation of blood. The cross-sectional area of the bridging portions between the spindle 470 and the impeller blades 410 has also been reduced to allow for further fluid flowthrough (relative to FIG. 13). The ratio of the bridging portions width to the apertures may be any ratio, but more preferably is in the range of 1:2 to 1:10. More particularly, the ratio of bridging portion width to aperture width is 1:3, or even more preferably, 1:4, or even more preferably, 1:5, or even more preferably, 1:6, or even more preferably, 1:7, or even more preferably, 1:8, or even more preferably, 1:9.

Further, the impeller 400 orientation may be reversed such that the impeller blades 410 convex shape side is the leading edge of the impeller blades 410, rather than the concave shape side being the leading edge of the impeller blades 410 as illustrated in FIG. 13. Reversing the direction of the impeller blades can impact the flow of fluids within the impeller housing 100 and may impart a desired flow to the fluid. This can be of particular advantage when the residence time of the fluid is to be increased or decreased. The leading edge and the following edge of the impeller blades 410 may be substantially the same shape or may be corresponding in shape. The shape of the following portion of the blade 410 may assist with imparting a laminar flow to the fluid, and may allow for a reduction in damage being imparted to the fluid.

The protrusion 800 may be an orientation marker which can be used to determine the placement or orientation of the device 10 when implanted. The protrusion 800 may alternatively be used to determine the effectiveness of a weld between the impeller housing 100 and the drive housing 200. The protrusion 800 in another embodiment may be a connection location for leads to the drive unit 200 and/or power source of the device.

The device 10 may be used to allow for providing a flow from the left atrium to the descending aorta. In this way the device 10 may be used to bypass at least one chamber of the heart. Further, having the flow from the atrium to the descending aorta may reduce stress on the heart and may improve patient health. The outlet 30 of the device 10 may be adapted to extend to any desired feature of the heart, such as the ventricle, ascending aorta, descending aorta, or any other desired configuration. The outlet may further be adapted, by way of having a differing cross sectional area at least at one portion, to increase or decrease the flow speed. The impeller may also ramp up or ramp down the rotational speed such that the flow of fluid can match more closely to the natural flowthrough velocity of the fluid in the heart, if the heart were undamaged.

In yet a further embodiment, the impeller 400 and/or pumping velocity is substantially velocity synchronised to the frequency of the heart. Alternatively, the synchronisation may be with respect to the contraction of a ventricle or the opening and/or closing of a valve of the heart, such as the mitral valve.

In yet another embodiment, the impeller 400 and/or pumping acceleration is substantially synchronised to the frequency of the heart. Alternatively, the acceleration synchronisation may be with respect to the contraction of a ventricle or the opening and/or closing of a valve of the heart, such as the mitral valve.

For example, opening the mitral valve (of the heart) may increase the acceleration of the impeller 400 (ramp up) and closing the mitral valve may decrease the acceleration of the impeller 400 (ramp down). The ramp up and ramp down of the impeller 400 may impact the flow of fluid through the device 10 and may cause a desired flow effect to be imparted to the fluid. The ramp up may impart a higher rotational velocity to the fluid such that the fluid is ejected further than when the impeller is ramping down or is ramped down. The ramp up and ramp down may be a ratio of the opening and closing of a valve.

For example, for every two openings of a valve the impeller 400 acceleration and/or velocity may be altered (ramp up or ramp down). It will be appreciated that any ratio may be used to impart a desired flow of fluid through the heart. Common ratios of ramp up to valve opening/closing may be 1:2, 1:3, 1:4 and 1:5. Preferably the ramp down of the impeller is mid-way between ramp ups of the impeller, although the ramp down may also be in a ratio of opening and closing of a valve. In this may the device 10 may provide for more comfortable use, particularly during sleep.

An algorithm signal may be used to allow for synchronisation between a heart movement and the impeller acceleration. The algorithm may be based on a pressure change sensed at or near to the inlet of the pump or the outlet of the pump, or the algorithm may be in relation to an ECG of a portion of the heart which may be sensed by a sensor. The sensor for the ECG may be attached to the device 10, or separate but may communicate sensed signals to the device 10.

Alternatively, opening the mitral valve may decrease the acceleration of the impeller 400 (ramp down) and closing the mitral valve may increase the acceleration of the impeller 400 (ramp up).

While not shown, the inlet and outlet may further comprise at least one directional flow means. Preferably, there is an inlet directional flow means and an outlet directional flow means. The inlet directional flow means may be directed towards the mitral valve such that blood entering the ventricle may more easily be directed to the device 10. The outlet directional flow means may be directed towards the aorta such that blood is more effectively delivery to the aorta.

Preferably the device causes a pressure differential such that blood is forced to flow from the device outlet and towards the aorta. This may cause a positive blood flow effect. Preferably the device is partially situated in the ventricle of the patient and the myocardium of the patient. More preferably, the device portion situated in the ventricle is proximal to the apex of the ventricle.

In yet another embodiment, the device 10 is a pulsatile ventricle assist device. The device 10 may allow for periodic or interval increases in the rotations per minute (RPM) of the impeller 400 which may correspond to the native pulse of a portion of a patient's heart.

FIG. 10 depicts an embodiment of the device 10 implanted partially in the left ventricle of a patient and a desirable fluid flow in the ventricle which allows for intake of blood from near the left atrium in the left ventricle through the inlet 20 and the fluid being pressured from the outlet 30 of the device 10 towards the aorta. Preferably the device 10 may cause a pressure differential to assist with the flow of blood through the heart in a pulsatile manner. Preferably the device 10 intakes blood in the ventricle, and improves the flow of blood in the ventricle. Preferably, the outlet of the device expels blood back into the same ventricle as the intake of blood.

It will be appreciated that the term “blood” may be replaced by the broader term “fluid” and the term blood is not limiting in any sense.

In an unillustrated embodiment, the hydrodynamic flow means may comprise a hydrodynamic thrust bearing or other suitable bearing for use with cardiovascular assist devices.

Typically, most VADs attempt to eliminate vortical flow of fluid into the device as this typically reduces the efficiency of the device. However, the device of the present disclosure may advantageously impart a vortical flow to fluid near to the inlet of the device which is subsequently flowed through into the cavity 120 with the impeller 400. In this way, damage to blood may be reduced as the fluid is imparted with a partial rotational flow prior to entering the cavity 120. The fluid flow may then be effected by the impeller 400 and ejected via the outlet 30 in a substantially laminar flow or a substantially irrotational flow. The transition of the flow is advantageous as this may allow for a device with improved efficiency, compared to devices which consider vortical flow to be undesired.

Optionally, in a further embodiment, the outlet 30 is configured to limit retrograde blood flow from the outlet 30 back to the inlet 20. The outlet 30 preferably comprises a flow direction means or is positioned in the direction of the apex of the ventricle. In this way the outlet 30 forces blood out of the apex and to flow is a desirable manner, such that shut down or collapse of the ventricle is avoided or delayed.

In yet another embodiment, the device 10 may include a radiopaque marker which may allow for viewing the orientation of the device when positioned in a heart. More particularly, the radiopaque marker preferably allows a physician to view the direction of the outlet and whether it needs to be repositioned to be in a desired direction. To allow for a desired alignment of the outlet 30, the preferred minimum distance of the inlet 20 relative to the outlet 30 is around 5 mm to 10 mm (5 millimetres to 10 millimetres). However, it is preferred that the distance is more than 10 mm if possible as this will further reduce retrograde flow experienced.

Biocompatible metals for use with at least one embodiment of the present disclosure may include; titanium, titanium and nickel alloys (including Nitinol), gold, silver, platinum, cobalt, chromium, or alloys comprising at least one of the aforementioned metals.

The following polymeric substances are examples of materials from which the embodiments may be constructed. For example, the polymers may include; Polyetheretherketone (PEEK), Fibre Reinforced Polymer (FRP), Polycarbonate (PC), Polysulphone (PS), Polyarethanes (PU), Polyether Polyurethanes (PEPU), Polycarbonate Urethane (PCU), Siloxane-Urethanes (SiU), Polyvinylchloride (PVC), Poly Vinylidene Fluoride (PVDF), Polyethylene (PE), Polypropylene (PP), Polymethylmethacrylate (PMMA), Acrylonitrile-Butadiene-Styrene Terpolymers (ABS), Polyesters (PET), Polyamides and/or Nylons (PA), Acetal Resins and/or Polyoxymethylene (AR), Polydimethylsiloxane (PDSM), Syndiotactic Polystyrene (SP), Aliphatic ether ketones (AEK), TOPAS™ (T), Metallocene PP (MPP), or any other suitable polymer.

Polyetheretherketone (PEEK)

An example of a polymeric material that may be used in the constructions of an embodiment is PEEK. It has a relatively high thermal stability compared with other thermoplastics. It typically retains high strength at elevated temperatures, and has excellent chemical resistance (being essentially inert to organics, and has a high degree of acid and alkali resistance). It has excellent hydrolytic stability and gamma radiation resistance. Therefore PEEK may be readily sterilised by different routes. It also shows good resistance to environmental stress cracking. It generally has excellent wear and abrasion resistance and a low coefficient of friction PEEK may incorporate glass and/or carbon fibre reinforcements which may enhance the mechanical and/or thermal properties of the PEEK material.

PEEK may be easily processed on conventional extrusion and injection moulding equipment. Post-annealing and other processes obvious to a person skilled in the art may be preferable. A polyaromatic, semicrystalline polymer may also be used in construction of an embodiment.

Other examples of this polymer include: Polyaryletherketone (PAEK) manufactured by Victrex and PEEK-OPTINMA LT™ which is a polymer grade with properties optimised for long-term implants. PEEK-OPTIMA LT™ is significantly stronger than traditional plastics currently available. Generally, PEEK may be able to withstand more aggressive environments and maintain impact properties over a broader range of temperatures than other polymers.

It has been shown that carbon fibre reinforced PEEK found to exhibit advantageous resistance to a saline environment at 37° C. designed to simulate human body conditions.

PEEK includes the significant advantage of generally supplying dimensional stability, when in use.

Fibre Reinforced Polymer (FRP)

Another example of a polymeric material that may be included within an embodiment of the present invention is FRP. FRPs are constructed of composites of PEEK and other polymers. PEEK may be reinforced with 30% short carbon fibres and which when subjected to saline soaking, was found to exhibit no degradation in mechanical properties. In contrast, a 30% short carbon fibre reinforced polysulphone composite has been found to show degraded mechanical properties due to the same saline soaking.

The fibre/matrix bond strength may significantly influence the mechanical behaviour of FRP composites. Interfacial bond strength durability is therefore particularly important in the development of FRP composites for implant applications, where diffused moisture may potentially weaken the material over time. Testing in physiologic saline at 37° C. showed that interfacial bond strengths in carbon fibre/polysulfone and carbon fibre/polyetheretherketone composites significantly decrease.

It should be noted that the fibre/matrix bond strength is known to strongly influence fracture behaviour of FRP composites.

Polycarbonate (PC)

Another example of polymer material that may be used in the construction of the preferred embodiments are PC resins. PC resins are widely used where transparency and general toughness are sought.

PC resins are intrinsically amorphous due to the large bulky bis-phenol component. This means that the polymer has a significantly high free volume and coupled with the polar nature of the carbonate group, the polymer can be affected by organic liquids and by water. PC resins are not as resistant to extremes in pH as PEEK however they are at least partially resistant.

PC resins generally have very low levels of residual monomers and so PC resins may be suitable for blood pump construction. PC resins generally have desirable mechanical and thermal properties, hydrophobicity and good oxidative stability. PC resins are desirably used where high impact strength is an advantage. PC resins also generally confer good dimensional stability, reasonable rigidity and significant toughness, at temperatures less than 140° C.

PC resins may be processed by all thermoplastic processing methods. The most frequently used process is injection moulding. Please note that it may be necessary to keep all materials scrupulously dry due to small but not negligible moisture pick-up of this resin. The melt viscosity of the resin is very high, and so processing equipment should be rugged. Processing temps of PC resins are relatively high generally being between approximately 230° C. and 300° C.

Polysulphone (PS)

Another example of a polymeric material that may be used to construct parts of an embodiment from is PS. PS has relatively good high temperature resistance, and rigidity. PC is generally tough but not notch-sensitive and is capable of use up to 140° C. It has excellent hydrolytic stability and is able to retain mechanical properties in hot and wet environments. PS is generally chemically inert.

PS is similar to PC resins but may be able to withstand more rigorous conditions of use. Additionally, PS is generally more heat resistant, and possesses a greater resistance to creep and better hydrolytic stability. PC has a high thermal stability generally due to bulky chemical side groups and rigid chemical main backbone chains. It is also generally resistant to most chemicals.

Injection moulding used for lower melt index grades, whilst extrusion and blow moulding is used to form components generally made of higher molecular weight PS.

Polyarethanes (PU)

Another example of a polymeric material that may be include within an embodiment of the present invention is PU. PU is one of the most biocompatible and haemocompatible polymeric materials. PU has the following properties: elastomeric characteristics; fatigue resistance; compliance and acceptance or tolerance in the body during healing; propensity for bulk and surface modification via hydrophilic/hydrophobic balance or by attachments of biologically active species such as anticoagulants or bio-recognisable groups. Bio-modification of PU may be possible through the use of a several antioxidants used in isolation or in combination. These antioxidants may include vitamin E, which may create materials which can endure in a patient's body for several years.

PU constitutes one of the few classes of polymers that include the properties of being generally highly elastomeric and biocompatible.

Polyether Polyurethanes (PEPU)

Another polymeric material that may be used in the construction of an embodiment is PEPU. PEPU generally has: relatively good flexural performance and acceptable blood compatibility.

Polycarbonate Urethane (PCU)

PCU may also provide another alternative polymeric material for the purpose of constructing an embodiment. PCU has significantly lower rates of water transmission or impermeability. This is due to inherently lower chain mobility of the carbonate structure in the soft segment phase. Additional impermeability to water vapour can be achieved by selecting a polyurethane polymer with high hard segment content, and aromatic rather than aliphatic di-isocyanate co-monomer, and a more hydrophobic surface.

PCU generally has oxidative stability of the carbonate linkage, which reduces the rate of biodegradation tremendously as compared to the polyether polyurethanes.

Siloxane-Urethanes (SiU)

SiU is another example of an alternative preferred polymeric material. SiU generally has a combination of properties including: fatigue strength, toughness, flexibility and low interaction with plasma proteins. However these polymers may be relatively soft.

Polyvinylchloride (PVC)

PVC is another example of an alternative preferred polymeric material. PVC is a relatively amorphous and rigid polymer which in the absence of plasticiser has a glass transition around Tg 75° C.-105° C. It is a cheap tough polymer which is extensively used with many types of filler and other additives. Although it has a high melt viscosity and therefore in theory is difficult to process, specialised methods have been established for several decades to compound this polymer efficiently.

Extraction-resistant grades of PVC are required for long-term blood compatibility. Plasticised PVC has been well established for blood bags and similar devices, and resin manufacturers can keep toxic residual monomer levels acceptably low (<1 ppm). However there is enormous social pressure to outlaw PVC despite scientific data which generally indicates that PVC is benign.

Poly Vinylidene Fluoride (PVDF)

PVDF is a polymer that possesses relatively good amounts of toughness and biocompatibility to be suitable for use in constructing an embodiment.

Polyethylene (PE)

PE is available in several major grades, including Low Density PE (LDPE), High Density PE (HDPE) and Ultra High Molecular Weight Grade PE (UHMWPE). However the UHMWPE may be likely to be the most suitable as it generally possesses relative toughness, low moisture absorption, and good overall chemical resistance.

Sintered and compression moulded UHMWPE has been well established for hip joints replacement. However further improvements appear necessary, as abrasive resistance and wear are not suitable for lengthy (>5-10 year) use. A major limitation of PE is thermal performance (melting point approximately 130° C.) and dimensional stability.

Polypropylene (PP)

Another suitable polymeric material is PP. PP is a versatile polymer that may possess a combination of features including: relative inertness, relatively good strength and good thermal performance. Depending on the grade, Tg ranges from 0° C. to −20° C. and the MPt is approximately 170° C. The most common grades are homo- and ethylene copolymers, the latter with improved toughness.

In addition, there have been many advances in reactor technology leading to grades which are either much softer than normal or much stiffer. For example, the Bassell Adstiff™ polymers made using Catalloy™ technology may be suitable and/or include desirable features for use in the manufacture of a blood pump. Generally, PP polymers lack the high melting point of PEEK, but this property is not generally desired.

Polymethylmethacrylate (PMMA)

PMMA is an amorphous material with good resistance to dilute alkalis and other inorganic solutions, and has been shown to be one of the most biocompatible polymers. Therefore, PMMA may include some of the desirable features and may be used in the construction of an embodiment of the present invention. Generally, PMMA easily machined with conventional tools, moulded, surface coated and plasma etched.

PMMA's may be susceptible to environmental stress cracking although this is usually associated with the use of organic solvents, not present in a patient's body and a blood pump working environment.

Acrylonitrile-Butadiene-Styrene Terpolymers (ABS)

ABS generally has relatively good surface properties including: hardness, good dimensional stability and reasonable heat resistance (Tg approximately 120° C.). The combination of the three monomers imparts stiffness (styrene), toughness (butadiene) and chemical resistance (acrylonitrile).

Other attributes of ABS may include: rigidity, high tensile strength and excellent toughness as well as excellent dimensional accuracy in moulding. ABS is generally unaffected by water, inorganic solvents, alkalis; acids; and alcohols. However certain hydrocarbon solvents, not usually present within the body of a patient or in the working environment of the blood pump, may cause softening and swelling on prolonged contact.

Polyesters (PET)

PET have become one of the largest growing thermoplastics over the past decade: volumes and prices are now approaching PE and PP. PET has a Tg around 75° C. and melting point of 275° C. It can vary from about 25% to 70% in crystallinity depending on the processing history of the polymer. Physical properties and chemical resistance are very dependent on crystallinity. PET may also have limited dimensional stability, as crystallisation can slowly increase after moulding. PET are generally tough, transparent, stiff and opaque.

Another class of PET with a Tg above 100° C. is currently available, this polymer is called Polyethylene Naphthenate (PEN). PET and PEN may both be suitable for use in the construction of a blood pump.

Polyamides and/or Nylons (PA)

PAs and Nylons are characterised by having excellent wear/frictional properties, high tensile impact and flexural strength and stiffness, good toughness and high melting points.

Some PAs may include relatively large hydrocarbon spacers between the amide groups. Examples of this type of PA include Nylon 11 and 12 which are generally more hydrophobic (water uptake <1%) than regular varieties of PAs. However the larger spacing leads to a loss in stiffness compared to the other polymers and thermal performance may also be compromised.

Fully aromatic polyamides including Kevlar™ and Nomexn5 are commercially available and have high stiffness and melting points. Semi-aromatic polyamides are made in Germany (eg Trogamid™ T) and France. These semi-aromatic polyamides generally have good transparency and chemical resistance.

Acetal Resins and/or Polyoxymethylene (AR)

AR may be used to construct any one of the preferred embodiments. This class of polymer is strong, hard, and abrasion resistant. It has been evaluated for joint replacement components and other long-term implants.

The acetal homo-polymer is prone to salt induced cracking, but copolymers with small amounts of a propylene oxide are possible. AR which contains formaldehyde may be of concern due to possible toxicity of formaldehyde.

Polydimethylsiloxane (PDSM)

PDSM may be used to construct any one of the preferred embodiments. This polymer is generally elastomeric. It may also be considered for use as either a biocompatible coating or a copolymer.

Copolymers based on PDMS and PU have been developed and PDMS/PC are commercially offered by General Electric as Lexan™ 3200. The latter is a fairly stiff transparent material with excellent UV performance.

Syndiotactic Polystyrene (SP)

SP may be used to construct any one of the preferred embodiments. SP is typically highly crystalline, little change in modulus occurs at the Tg of 100° C., and retention of properties is fairly high to the melting point of over 250° C. Many grades may be fibre reinforced, to filer reduce the change in modulus at the Tg. Being a hydrocarbon with no hetero atoms, the polymer may be hydrophobic and inert.

Aliphatic Ether Ketones (AEK)

AEK may be used to construct any one of the preferred embodiments. Processing and mechanical performance are similar, but this polymer shows improved high temperature aging behaviour and little notch sensitivity. Unfortunately the material lacked distinctiveness and is no longer produced.

TOPAS™ (T)

T may be used to construct any one of the preferred embodiments. This class of co-polymer is made by Ticona in Germany. It generally comprises ethylene and norbomadene, with the Tg being controlled by monomer ratio. It is a hydrocarbon alternative to polycarbonate, and is generally suitable for medical fittings and devices. Its Tg is over approximately 130° C. and it is generally transparent with the co-monomer inhibiting crystallisation of the ethylene segments.

Metallocene PP (MPP)

MPP may be used to construct any one of the preferred embodiments MPP is manufactured by Exxon to compete with existing PP. It has a much narrower molecular weight distribution (polydispersity) because it is oligomer-free.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

1. A ventricle assist device, the device comprising;
a device body with a housing, an inlet and an outlet;
a centrifugal pump disposed in a portion of the housing;
the inlet adapted to allow a flow of blood into the device body housing and an outlet adapted to allow the flow of blood from the device body housing; and
wherein the rotational acceleration of the centrifugal pump is substantially synchronised to at least one of opening a heart valve and closing the heart valve.

2. The ventricle assist device of claim 1, wherein the heart valve is a mitral valve.

3. The device as claimed in claim 1, wherein an impeller is at least partially positioned in the ventricle.

4. The device as claimed in claim 1, wherein the device causes a pressure differential in the ventricle.

5. The device as claimed in claim 4, wherein the pressure differential is adapted to direct a flow of blood towards the aorta.

6. The device as claimed in claim 1, wherein the inlet is disposed relatively perpendicular to the outlet.

7. The device as claimed in claim 1, wherein a relative distance between the inlet and the outlet is at least 10 mm.

8. The device as claimed in claim 1, wherein an upper end of the housing is conically tapered to the inlet.

9. The device as claimed in claim 1, wherein a battery is disposed in the housing.

10. The device as claimed in claim 1, wherein the device can effect a vortical flow adjacent to the inlet.

11. The device as claimed in claim 1, wherein the device is adapted to eject a laminar flow of fluid.

12. The device as claimed in claim 1, wherein the housing comprises an impeller housing and a drive unit housing.

13. The device as claimed in claim 12, wherein the drive unit housing is adapted to house a drive unit of the centrifugal pump.

14. The device as claimed in claim 12, wherein the impeller housing comprises an impeller of the centrifugal pump.

15. The device as claimed in claim 1, wherein the outlet is directed towards the apex of the ventricle.

16. The device as claimed in claim 1, wherein the impeller comprises a radiopaque marker.
